(19) United States
(12) Patent Application Publication
(10) Pub. No.: US 2025/0216412 A1
(43) Pub. Date: Jul. 3, 2025

(54) MEDICAL IMAGING SYSTEM, AND VIBRATION DETECTION METHOD AND VIBRATION DETECTION APPARATUS THEREOF

(71) Applicant: GE Precision Healthcare LLC, Waukesha, WI (US)

(72) Inventors: Kehao Zhang, Beijing (CN); Ruiyu Wang, Beijing (CN)

(21) Appl. No.: 18/988,791

(22) Filed: Dec. 19, 2024

(30) Foreign Application Priority Data
Dec. 27, 2023 (CN) 202311823396.5

(51) Int. Cl. G01P 15/18; A61B 6/00; G01P 15/125

(52) U.S. Cl. CPC G01P 15/18; A61B 6/4435; G01P 15/125

(57) ABSTRACT

The present invention relates to a medical imaging system, and a vibration detection method and a vibration detection apparatus thereof. The vibration detection apparatus may include two detection plates at an angle to each other, a sensing unit being mounted on each detection plate, and the sensing units being used to sense vibrations in two directions independently of each other. The medical imaging system may include a gantry, including a fixed portion and a rotatable rotating portion mounted on the fixed portion; an example vibration detection apparatus which is mounted on the gantry. Also provided in the present invention is a vibration detection method corresponding to the example vibration detection apparatus and the example medical imaging system. According to the present invention, a vibration sampling frequency bandwidth can be significantly increased, and high-precision low-noise sampling can be performed.

### CROSS-REFERENCE TO RELATED APPLICATIONS

This application claims priority to Chinese Application No. 202311823396.5, filed on Dec. 27, 2023, the disclosure of which is incorporated herein by reference in its entirety.

### TECHNICAL FIELD

The present invention relates generally to the field of medical devices, and more particularly to a medical imaging system, and a vibration detection method and a vibration detection apparatus thereof.

### BACKGROUND

Medical imaging systems are generally used to scan an examination subject (e.g., a patient) to acquire, in a non-invasive manner, images reflecting the internal structure or function of the scan subject, for example, a computed tomography (CT) device. Currently widely used third generation CT devices include a gantry consisting of a stationary portion and a rotating portion. The rotating portion is mounted on the stationary portion, and rotates at a high speed during scanning. Balance monitoring is performed by mounting a vibration detection apparatus on the gantry.

In practice, however, a sampling bandwidth of existing vibration detection apparatuses cannot achieve satisfactory sampling precision and a satisfactory noise level.

Therefore, there is a great need for a novel vibration detection technique that is applied to a medical imaging system and that is capable of improving sampling precision and noise level while increasing a sampling frequency bandwidth.

### SUMMARY

The present invention aims to overcome the above and/or other problems in the prior art. With the novel design, a vibration detection method and a vibration detection apparatus of the present invention can significantly increase a sampling frequency bandwidth, and perform precision low-noise sampling, and accordingly, a medical imaging system can perform accurate vibration detection and balance monitoring.

According to a first aspect of the present invention, provided is a vibration detection apparatus for a medical imaging system, including two detection plates at an angle to each other, a sensing unit being mounted on each detection plate, and the sensing units being used to sense vibrations in two directions independently of each other.

According to a second aspect of the present invention, also provided correspondingly is a vibration detection method for a medical imaging system, including the following steps: respectively independently sensing vibrations in two directions via sensing units mounted on two detection plates at an angle to each other.

The sensing units are respectively mounted on the two detection plates at an angle to each other, and can sense vibrations in two directions independently of each other, thereby lowering a noise level while significantly improving sampling precision. In addition, the above design allows a sensing unit having a larger sampling frequency bandwidth to be used, thereby increasing the sampling bandwidth by several thousand times. Furthermore, the sensing units are provided on the two detection plates, so that the two sensing units are prevented from interfering with each other to affect accuracy of vibration monitoring and balance detection.

Further, the two detection plates are configured to be perpendicular to each other, so that the two sensing units perform detection while being decoupled from each other.

According to a third aspect of the present invention, provided is a medical imaging system, which may include a gantry and the above vibration detection apparatus according to the present invention. The gantry may include a fixed portion and a rotatable rotating portion mounted on the fixed portion. Accordingly, in the vibration detection method of the present invention, the two detection plates may be mounted on a gantry of a medical imaging system to detect rotational vibrations of a rotating portion of the gantry.

Since the novel vibration detection apparatus of the present invention is used, the sampling precision achieved thereby completely satisfies requirements on detection of rotational vibrations of the rotating portion of the gantry of the medical imaging system, and a signal-to-noise ratio is significantly increased due to greater sensitivity. In the medical imaging system of the present invention, a sampling rate of gantry balance data is greatly improved, which greatly facilitates monitoring of the gantry balance, thereby ensuring that the medical imaging system acquires high-quality images.

In the medical imaging system of the present invention, the two detection plates may be mounted on the top of the fixed portion to respectively detect vibration signals in an X-axis direction and a Z-axis direction. Accordingly, in the vibration detection method of the present invention, the two detection plates may be mounted on the top of the fixed portion to respectively detect vibration signals in an X-axis direction and a Z-axis direction. A Z axis is perpendicular to a plane defined by an X axis and a Y axis, and the plane defined by the X axis and the Y axis is parallel to a plane of rotation of the rotating portion.

Since the vibration signal at the top of the fixed portion is the strongest, the two detection plates are mounted on the top of the fixed portion to further facilitate signal detection.

Each of the two detection plates may further include: a first stage differential amplification module and a second stage low-pass module. The first stage differential amplification module is used to amplify a signal sensed by the sensing unit. The second stage low-pass module is used to perform low-pass filtering on a signal outputted by the first stage differential amplification module. Accordingly, the above vibration detection method may further include the following steps: amplifying a signal sensed by the sensing unit; and performing low-pass filtering on the amplified signal. Thus, extraction of a vibration signal caused by rotation of the rotating portion is facilitated.

In the above medical imaging system, the first stage differential amplification module may include in sequence a low-pass filter, a high-pass filter, and a signal amplifier. Accordingly, in the above vibration detection method, the sensed signal may be amplified by causing the signal sensed by the sensing unit to pass in sequence through a low-pass filter, a high-pass filter, and a signal amplifier.

In the above medical imaging system, the second stage low-pass module may include one or more low-pass filters. Accordingly, in the above vibration detection method, the amplified signal may undergo low-pass filtering by causing the amplified signal to pass through one or more low-pass filters.

In the above medical imaging system, each of the two detection plates may further include: a third stage output amplification module, used to perform output amplification on the signal that has undergone low-pass filtering by the second stage low-pass module. Accordingly, the above vibration detection method may further include the following step: performing output amplification on the signal that has undergone low-pass filtering. Thus, noise can be further removed, thereby further facilitating extraction of a vibration signal caused by rotation of the rotating portion.

In the above medical imaging system, the third stage output amplification module may include, in sequence, a high-pass filter, a signal amplifier, and a current limiter. Accordingly, in the above vibration detection method, output amplification may be performed on the signal that has undergone low-pass filtering by causing the signal that has undergone low-pass filtering to pass in sequence through a high-pass filter, a signal amplifier, and a current limiter.

Other features and aspects of the present invention will become clearer via the detailed description provided below with reference to the accompanying drawings.

### BRIEF DESCRIPTION OF THE DRAWINGS

The present invention can be better understood by means of the description of the exemplary embodiments of the present invention in conjunction with the drawings, in which:

FIG. 1 is a schematic diagram of a vibration detection apparatus according to the present invention;

FIG. 2 is a schematic diagram of an embodiment of a vibration detection apparatus according to the present invention;

FIG. 3 is a schematic diagram of another embodiment of a vibration detection apparatus according to the present invention;

FIG. 4 is a schematic diagram of a medical imaging system according to the present invention;

FIG. 5 is a schematic diagram of an embodiment of a medical imaging system according to the present invention;

FIG. 6(a) and FIG. 6(b) are schematic diagrams of other embodiments of a medical imaging system according to the present invention;

FIG. 7 shows a schematic diagram of a vibration detection apparatus in another embodiment of a medical imaging system according to the present invention;

FIG. 8 is an example of signal processing in a detection plate in the embodiment shown in FIG. 7; and

FIG. 9 is a schematic diagram of a vibration detection apparatus in still another embodiment of a medical imaging system according to the present invention.

### DETAILED DESCRIPTION

The present invention will be further described below with reference to specific embodiments and the accompanying drawings. More details are set forth in the following description in order to facilitate thorough understanding of the present invention, but it will be apparent that the present invention can be implemented in many other forms other than those described herein, and those skilled in the art can, without departing from the spirit of the present invention, make similar alterations and modifications according to practical applications. Therefore, the scope of protection of the present invention should not be limited by the contents of the specific embodiments.

Unless defined otherwise, technical terms or scientific terms used in the claims and description should have the usual meanings that are understood by those of ordinary skill in the technical field to which the present invention belongs. Terms such as “first”, “second”, and similar terms used in the description and claims of the present application do not denote any order, quantity, or importance, but are only intended to distinguish different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

According to an embodiment of the present invention, a vibration detection apparatus is provided.

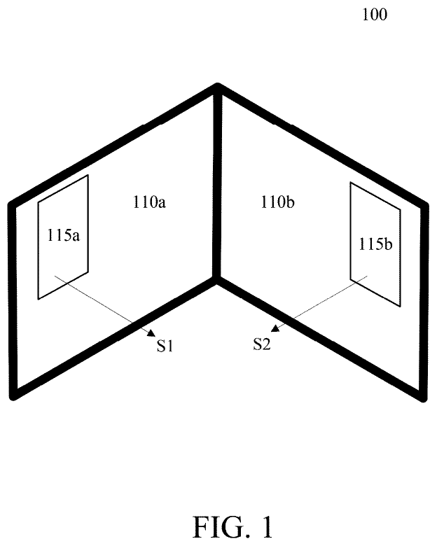

FIG. 1 is a schematic diagram of a vibration detection apparatus 100 according to the present invention. As shown in FIG. 1, the vibration detection apparatus 100 may include two detection plates 110a and 110b at an angle to each other. Sensing units 115a and 115b are respectively mounted on the detection plates 110a and 110b, to sense vibrations in two directions (for example, vibrations in S1 and S2 directions in FIG. 1, the two directions being respectively perpendicular to the sensing units 115a and 115b) independently of each other.

The sensing unit may be, for example, an acceleration sensor. When an object vibrates in a certain direction, the acceleration sensor senses corresponding acceleration of the object, so that intensity of the vibration can be calculated. Acceleration sensors that may be used include a piezoresistive accelerometer, a capacitive accelerometer, a tunneling accelerometer, an optical accelerometer, a piezoelectric accelerometer, and the like. The acceleration sensors of the two sensing units 115a and 115b may be alternating current (AC) coupled so as to have the same measurement reference. Finally, the acceleration sensor converts a sensed acceleration signal into a direct current voltage for output. For example, when the relationship between the acceleration and the direct current voltage is 1.35 V/g, the acceleration sensor converts the sensed acceleration signal of 1 g into a direct current voltage of 1.35V for output. Similarly, if the acceleration signal is 2 g, the direct current voltage of 2.7V is output.

In an exemplary embodiment of the present invention, the vibration detection apparatus employs a three-dimensional design in which the sensing units are respectively mounted on the two detection plates that are at an angle to each other. Compared with the existing two-dimensional design in which two acceleration sensors are mounted on one detection plate, the vibration detection apparatus “with the increased dimensionality” of the present invention breaks the spatial limitation of the existing design. The sensing units on the two detection plates decoupled from each other can perform respective detection without interfering with each other, and in addition, such a design makes it possible to allow sensing units with larger sampling frequency bandwidths to be used, thereby dramatically increasing the existing sampling frequency bandwidth of several Hz to several thousand Hz. Such a novel vibration detection apparatus “with the increased dimensionality” of the present invention has greater sensitivity, and accordingly, a signal-to-noise ratio can also be significantly increased. As compared with the prior art in which when one sensor loosens, the other sensor vibrates accordingly, the two detection plates in the vibration detection apparatus of the present invention are independent from each other, so that when one detection plate or the sensing unit thereon loosens or is faulty, the other detection plate and normal operation of the sensing unit mounted thereon are not affected.

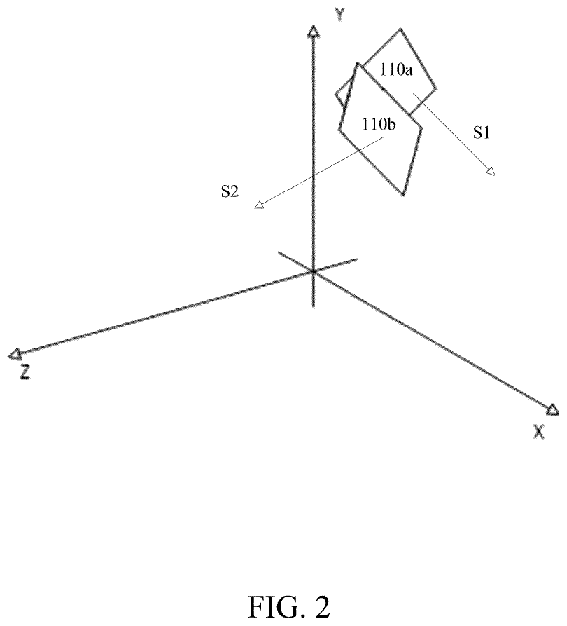

The detection plates 110a and 110b may be at any angle to each other, and the corresponding sensing units 115a and 115b can sense vibrations in the S1 direction and the S2 direction. In some embodiments, during vibration detection, directions in which vibrations are sensed may be different from the S1 direction and the S2 direction respectively perpendicular to the detection plates 110a and 110b as shown in FIG. 1. For example, as shown in FIG. 2, the directions in which vibrations need to be sensed are X-axis and Z-axis directions. In this case, X-axis direction and Z-axis direction components, i.e., X₁₁₅ₐ and Z₁₁₅ₐ as well as X_{115b} and Z_{115b}, may be respectively extracted from output signals of the sensing units 115a and 115b, and may then be respectively superimposed to acquire a vibration signal X₁₁₅ₐ+X_{115b} in an X axis and a vibration signal Z₁₁₅ₐ+Z_{115b} in a Z axis.

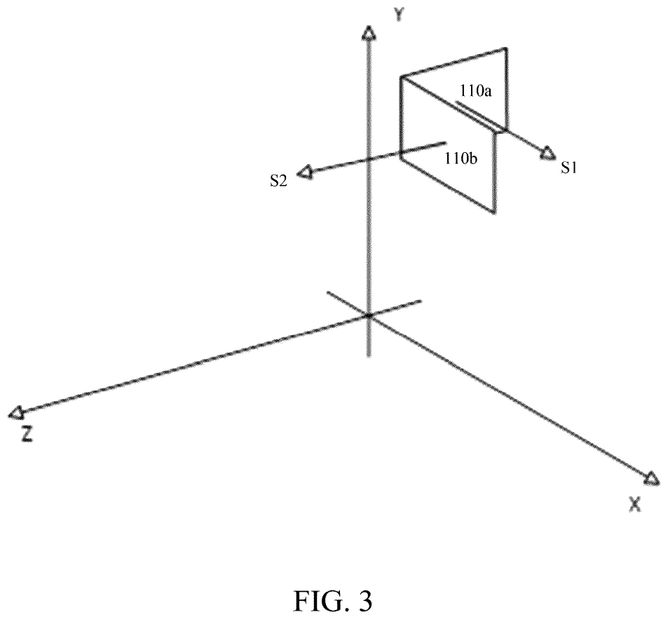

Optionally, the detection plates 110a and 110b may be configured to be perpendicular to each other as shown in FIG. 3, so that the sensing units 115a and 115b may perform detection while being further decoupled from each other (i.e., without interfering with each other).

According to an embodiment of the present invention, a medical imaging system is further provided.

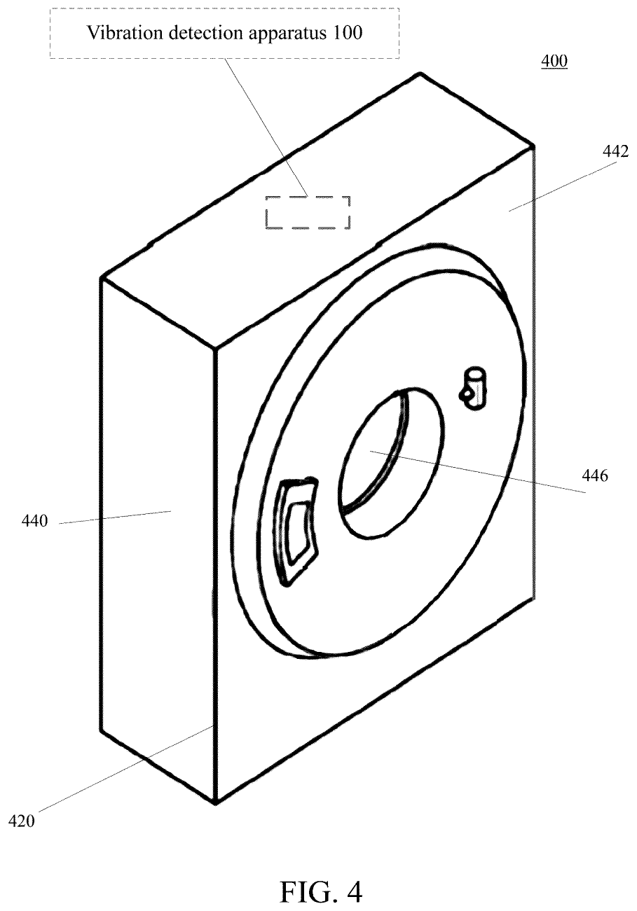

FIG. 4 is a schematic diagram of a medical imaging system 400 according to the present invention. As shown in FIG. 4, the medical imaging system 400 may include a housing 420, a gantry 440 mounted in the housing 420 and having a scanning hole, and the above vibration detection apparatus 100. The gantry 440 includes a fixed portion 442 and a rotatable rotating portion 446 mounted on the fixed portion 442. In FIG. 4, a mounting position of the vibration detection apparatus 100 is merely indicated by a dotted line. It can be understood that the vibration detection apparatus 100 may be mounted on the top of the fixed portion 442 as shown in FIG. 4, or may be mounted on a leg portion of the fixed portion 442, or may be mounted in another position on the gantry 440.

The medical scanning system of the present invention employs the vibration detection apparatus of the present invention. The sensing units perform respective detection without interfering with each other, thereby lowering a noise level while significantly improving sampling precision. In addition, sensing units with larger sampling frequency bandwidths are allowed to be used, so that a vibration signal can be measured by using a larger sampling bandwidth of up to several thousand Hz. The sampled data can greatly improve convenience and accuracy of calculating the dynamic and static balance of the gantry, thereby reliably ensuring balance monitoring of the gantry, and facilitating acquisition of high-quality images by the medical imaging system.

Acceleration sensors that may be used in the sensing units 115a and 115b in the vibration detection apparatus 100 include one or a plurality of a piezoresistive accelerometer, a capacitive accelerometer, a tunneling accelerometer, an optical accelerometer, a piezoelectric accelerometer, or the like. The capacitive accelerometer is preferably used in the medical scanning system of the present invention to perform vibration detection because the capacitive accelerometer has both the features of high sensitivity and low noise, and is more suitable for monitoring rotational vibrations of the gantry.

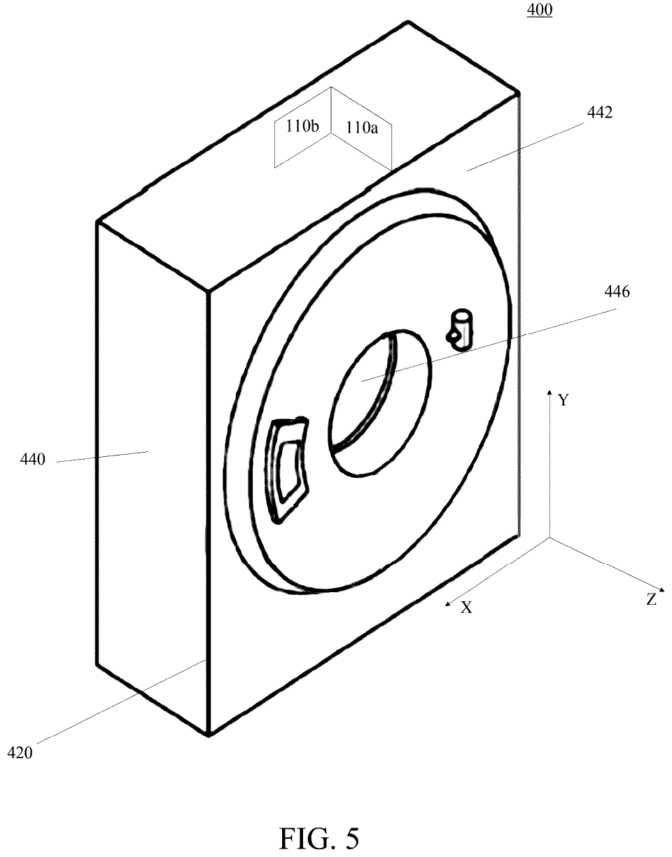

Optionally, in the above medical imaging system 400, as shown in FIG. 5, the two detection plates 110a and 110b may be mounted on the top of the fixed portion 442 to respectively detect vibration signals in an X-axis direction and a Z-axis direction, wherein a Z axis is perpendicular to a plane defined by an X axis and a Y axis, and the plane defined by the X axis and the Y axis is parallel to a plane of rotation of the rotating portion 446.

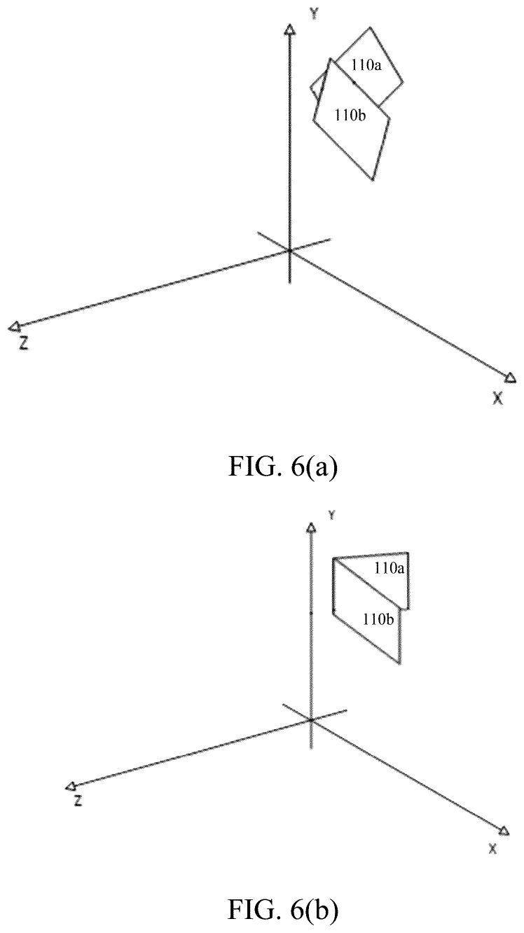

Taking a CT machine as an example, when the CT machine is operating, the rotating portion in the gantry constantly rotates parallel to an XOY plane in FIG. 5, and the rotation causes the gantry to vibrate in the X-axis direction and the Z-axis direction in FIG. 5, thereby changing the dynamic balance and/or static balance of the CT machine, which affects quality of final imaging. Therefore, it is necessary to detect the vibrations of the gantry in the X-axis direction and the Z-axis direction caused by the rotation of the rotating portion. When the detection plates 110a and 110b are mounted on the top of the fixed portion 442 as shown in FIG. 5, vibration signals that can be sensed by the sensing units 115a and 115b are the strongest, thereby facilitating acquisition of the vibration signals. The detection plates 110a and 110b in FIG. 5 are respectively perpendicular to the X axis and the Z axis, so that signals sampled by the sensing units 115a and 115b are directly the vibration signals in the X-axis direction and the Z-axis direction. However, it can be understood that in other embodiments, at least one of the detection plates 110a and 110b forms a tilt angle (not a 90° angle) with the corresponding X axis or Z axis. For example, the detection plates 110a and 110b are not perpendicular to each other (as shown in FIG. 6(a)), or the detection plates 110a and 110b are perpendicular to each other, but are not respectively perpendicular to the X axis and the Z axis (as shown in FIG. 6(b)). In this case, as described above, X-axis direction and Z-axis direction components, i.e., X₁₁₅ₐ and Z₁₁₅ₐ as well as X_{115b} and Z_{115b}, may be respectively extracted from output signals of the sensing units 115a and 115b, and may then be respectively superimposed to acquire a vibration signal X₁₁₅ₐ+X_{115b} in the X axis and a vibration signal Z₁₁₅ₐ+Z_{115b} in the Z axis.

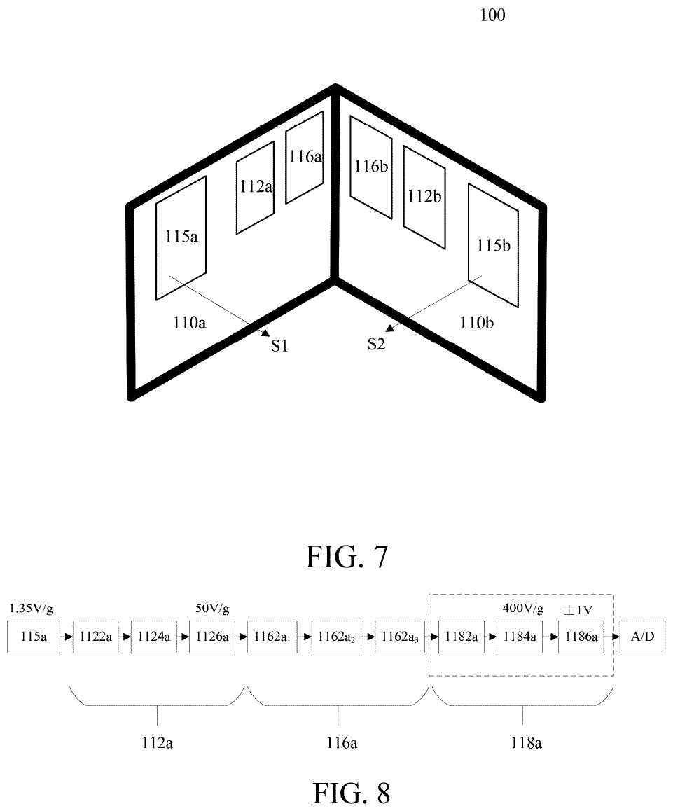

Optionally, for the above medical imaging system 400, the detection plates 110a and 110b may further respectively include first stage differential amplification modules 112a and 112b and second stage low-pass modules 116a and 116b, as shown in FIG. 7. The first stage differential amplification module 112a may be used to amplify an output signal of the sensing unit 115a, and the second stage low-pass module 116a may be used to low-pass filter the signal amplified by the first stage differential amplification module 112a. Similarly, the first stage differential amplification module 112b may be used to amplify an output signal of the sensing unit 115b, and the second stage low-pass module 115b may be used to low-pass filter the signal amplified by the first stage differential amplification module 112b.

Still taking the CT machine as an example, since a fan in the gantry also generates vibrations (the frequency of the vibrations is large) when operating, the signals outputted by the sensing units 115a and 115b may be processed to better extract the vibrations caused by the rotation of the rotating portion. By means of the above processing performed by the first stage differential amplification modules and the second stage low-pass modules, a direct current noise component in the signal can be further removed, and the signal corresponding to the vibrations caused by the rotation of the rotating portion (the frequency of which is smaller than that of the vibrations caused by the fan) can be effectively extracted.

Optionally, the first stage differential amplification modules 112a and 112b may each include in sequence a low-pass filter, a high-pass filter, and a signal amplifier.

Optionally, the second stage low-pass modules 116a and 116b may each include one or more low-pass filters.

Via the above first stage differential amplification modules and second stage low-pass modules of the present invention, it is possible to capture a vibration signal of a specific frequency band generated by the gantry.

FIG. 8 shows an example of signal processing in the above detection plate 110a, in which the sensing unit 115a is an acceleration sensor, and the low-pass filter 1162a in the second stage low-pass module 116a includes three low-pass filters 1162a₁ to 1162a₃. As shown in FIG. 8, the acceleration sensor 115a senses, for example, a vibration signal 1.35 V/g (that is, when acceleration is 1 g, a corresponding direct current voltage is 1.35V). The vibration signal passes through a low-pass filter 1122a, and high-frequency noise therein is filtered out or removed. For example, only a signal lower than or equal to K1 Hz can pass. The vibration signal then passes through a high-pass filter 1124a, and a low-frequency direct current component therein is filtered out or removed. For example, only a signal higher than or equal to K2 Hz can pass. Then, the vibration signal passes through a signal amplifier 1126a so as to be converted from a differential signal into a single-ended signal. Finally, the vibration signal is output from the signal amplifier 1126a. For example, the output vibration signal is 50 V/g (that is, when acceleration is 1 g, a corresponding direct current voltage is 50V). Thereafter, the vibration signal 50 V/g (with the frequency between K2 Hz and K1 Hz) outputted by the first stage differential amplification module 112a passes in sequence through the second stage low-pass filters 1162a₁ to 1162a₃, so as to further remove high-frequency noise in the signal.

It can be understood that signal processing in the above detection plate 110b is similar.

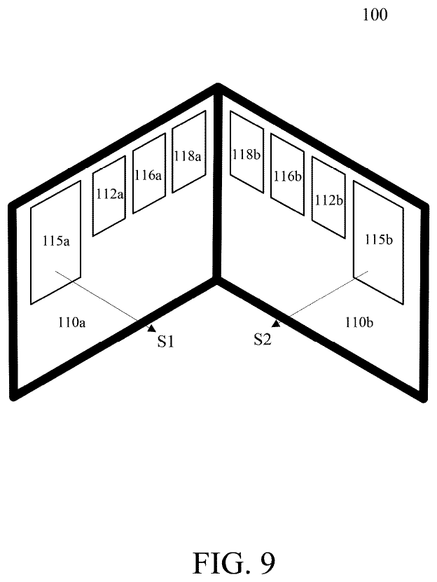

Optionally, the detection plates 110a and 110b may further respectively include third stage output amplification modules 118a and 118b, as shown in FIG. 9. The third stage output amplification module 118a may be used to amplify the signal outputted by the second stage low-pass module 116a. Similarly, the third stage output amplification module 118b may be used to amplify the signal outputted by the second stage low-pass module 116b. By means of the processing performed by the third stage output amplification modules, a direct current noise component in the signal can be further removed, and the signal corresponding to the vibrations caused by the rotation of the rotating portion can be more effectively extracted. However, it can be understood that, in some embodiments, signals of other vibration sources other than the rotational vibrations of the rotating portion have been effectively filtered out or removed from the signals processed by the first stage differential amplification modules and the second stage low-pass modules, and the third stage output amplification modules may not be required.

Optionally, the third stage output amplification modules 118a and 118b may each include in sequence a high-pass filter, a signal amplifier, and a current limiter.

The signal processing in the above detection plate 110a will still be described by using the example in FIG. 8. The vibration signals outputted by the second stage low-pass filters 1162a₁ to 1162a₃ are transmitted to a high-pass filter 1182a to further remove a low-frequency direct current component from the vibration signals, and are then transmitted to a signal amplifier 1184a of which a gain is, for example, 8, so that the vibration signals output therefrom are 400 V/g (that is, when acceleration is 1 g, a corresponding direct current voltage is 400V). The vibration signal outputted by the signal amplifier 1184a is further transmitted to a current limiter 1186a that can prevent a finally output voltage from incurring damage or faulty to other processing circuits that are subsequently used to calculate the dynamic and static balance and the like. For example, a current limit voltage is ±1V. That is, the voltage finally output from the third stage output amplification module 118a does not exceed ±1V.

According to an embodiment of the present invention, also provided correspondingly is a vibration detection method, which may include the following step S0: respectively independently sensing vibrations in two directions via sensing units mounted on two detection plates at an angle to each other.

Optionally, the above two detection plates may be perpendicular to each other.

Optionally, the two detection plates may be mounted on a gantry of a medical imaging system to detect rotational vibrations of a rotating portion of the gantry.

Optionally, the two detection plates may be mounted on the top of a fixed portion of the gantry to respectively detect vibration signals in an X-axis direction and a Z-axis direction, wherein a Z axis is perpendicular to a plane defined by an X axis and a Y axis, and the plane defined by the X axis and the Y axis is parallel to a plane of rotation of the rotating portion.

Optionally, the vibration detection method may further include steps S1 and S2. In S1, a signal sensed by the sensing unit is amplified. In S2, low-pass filtering is performed on the amplified signal.

Optionally, in step S1, the sensed signal may be amplified by causing the signal sensed by the sensing unit to pass in sequence through a low-pass filter, a high-pass filter, and a signal amplifier.

Optionally, in step S2, the amplified signal may undergo low-pass filtering by causing the amplified signal to pass through one or more low-pass filters.

Optionally, the vibration detection method may further include step S3: performing output amplification on the signal that has undergone low-pass filtering.

Optionally, in step S3, the output amplification may be performed on the signal that has undergone low-pass filtering by causing the signal that has undergone low-pass filtering to pass in sequence through a high-pass filter, a signal amplifier, and a current limiter.

The above vibration detection method completely corresponds to the vibration detection apparatus and the medical imaging system according to the present invention described above. The above many design concepts and details applicable to the vibration detection apparatus and the medical imaging system of the present invention are also applicable to the above vibration detection method, and the same advantageous technical effects can be achieved, so that the detailed description thereof is omitted here.

Various aspects of the present invention have been described above via some exemplary embodiments. However, it should be understood that various modifications can be made to the exemplary embodiments described above without departing from the spirit and scope of the present invention. For example, an appropriate result can be achieved if the described techniques are performed in a different order and/or if the components of the described system, architecture, apparatus, or circuit are combined in other manners and/or replaced or supplemented with additional components or equivalents thereof; accordingly, the modified other embodiments also fall within the protection scope of the claims.

1. A vibration detection apparatus for a medical imaging system, comprising:
two detection plates at an angle to each other, a sensing unit being mounted on each detection plate, and the sensing units being used to sense vibrations in two directions independently of each other.

2. The vibration detection apparatus according to claim 1, wherein the two detection plates are perpendicular to each other.

3. A medical imaging system, comprising:
a gantry, including a fixed portion and a rotatable rotating portion mounted on the fixed portion; and
a vibration detection apparatus mounted on the gantry, wherein the vibration detection apparatus includes two detection plates at an angle to each other, a sensing unit being mounted on each detection plate, and the sensing units being used to sense vibrations in two directions independently of each other.

4. The medical imaging system according to claim 3, wherein the two detection plates are mounted on the top of the fixed portion to respectively detect vibration signals in an X-axis direction and a Z-axis direction, wherein a Z axis is perpendicular to a plane defined by an X axis and a Y axis, and the plane defined by the X axis and the Y axis is parallel to a plane of rotation of the rotating portion.

5. The medical imaging system according to claim 3, wherein each of the two detection plates further includes:
a first stage differential amplification module, used to amplify a signal sensed by the sensing unit; and
a second stage low-pass module, used to perform low-pass filtering on a signal outputted by the first stage differential amplification module.

6. The medical imaging system according to claim 5, wherein the first stage differential amplification module includes, in sequence, a low-pass filter, a high-pass filter, and a signal amplifier.

7. The medical imaging system according to claim 5, wherein the second stage low-pass module includes one or more low-pass filters.

8. The medical imaging system according to claim 5, wherein each of the two detection plates further includes:
a third stage output amplification module, used to perform output amplification on the signal that has undergone low-pass filtering by the second stage low-pass module.

9. The medical imaging system according to claim 8, wherein the third stage output amplification module includes, in sequence, a high-pass filter, a signal amplifier, and a current limiter.

10. A vibration detection method for a medical imaging system, the method comprising:
respectively independently sensing vibrations in two directions via sensing units mounted on two detection plates at an angle to each other.

11. The vibration detection method according to claim 10, wherein the two detection plates are perpendicular to each other.

12. The vibration detection method according to claim 10, wherein the two detection plates are mounted on a gantry of a medical imaging system to detect rotational vibrations of a rotating portion of the gantry.

13. The vibration detection method according to claim 12, wherein the two detection plates are mounted on the top of a fixed portion of the gantry to respectively detect vibration signals in an X-axis direction and a Z-axis direction, wherein a Z axis is perpendicular to a plane defined by an X axis and a Y axis, and the plane defined by the X axis and the Y axis is parallel to a plane of rotation of the rotating portion.

14. The vibration detection method according to claim 12, further including:
amplifying a signal sensed by the sensing unit; and
performing low-pass filtering on the amplified signal.

15. The vibration detection method according to claim 14, wherein the sensed signal is amplified by causing the signal sensed by the sensing unit to pass in sequence through a low-pass filter, a high-pass filter, and a signal amplifier.

16. The vibration detection method according to claim 14, wherein the amplified signal undergoes low-pass filtering by causing the amplified signal to pass through one or more low-pass filters.

17. The vibration detection method according to claim 12, further including performing output amplification on the signal that has undergone low-pass filtering.

18. The vibration detection method according to 17, wherein output amplification is performed on the signal that has undergone low-pass filtering by causing the signal that has undergone low-pass filtering to pass in sequence through a high-pass filter, a signal amplifier, and a current limiter.
